(19) United States
(12) Patent Application Publication
(10) Pub. No.: US 2019/0225685 A1
(43) Pub. Date: Jul. 25, 2019

(54) ANTI-DLL3 ANTIBODY DRUG CONJUGATES AND METHODS OF USE

(71) Applicant: ABBVIE STEMCENTRX LLC, NORTH CHICAGO, IL (US)

(72) Inventors: KUMIKO ISSE, BURLINGAME, CA (US); LAURA SAUNDERS, SAN FRANCISCO, CA (US)

(73) Assignee: ABBVIE STEMCENTRX LLC, NORTH CHICAGO, IL (US)

(21) Appl. No.: 16/256,628

(22) Filed: Jan. 24, 2019

Related U.S. Application Data
(60) Provisional application No. 62/621,245, filed on Jan. 24, 2018.

(51) Int. Cl. C07K 16/28; A61P 35/00; A61K 47/68; A61K 31/5517; A61K 47/65; A61K 47/14

(52) U.S. Cl. CPC C07K 16/28; A61P 35/00; A61K 47/6803; A61K 47/14; A61K 47/6851; A61K 47/65; A61K 31/5517

(57) ABSTRACT

Provided are novel anti-DLL3 antibodies and antibody drug conjugates, and methods of using such anti-DLL3 antibodies and antibody drug conjugates to treat brain cancer.

### CROSS REFERENCED APPLICATIONS

This application claims the benefit of U.S. Provisional Application No. 62/621,245 filed on Jan. 24, 2018, which is incorporated herein by reference in its entirety.

### SEQUENCE LISTING

This application contains a sequence listing that has been submitted in ASCII format via EFS-Web and is hereby incorporated by reference in its entirety. Said ASCII copy, created on Jan. 24, 2019 is named ABV12427USO1_Sequence_Listing and is 6 KB (6,184 bytes) in size.

### FIELD OF THE INVENTION

This application generally relates to administering anti-DLL3 antibodies or immunoreactive fragments thereof, including antibody drug conjugates (ADCs) comprising the same for the treatment, diagnosis or prophylaxis of neurological malignancies and any recurrence or metastasis thereof. In selected embodiments the invention provides for the administration of such anti-DLL3 antibodies or antibody drug conjugates for the treatment of glioma, including diffuse and/or low grade glioma.

### BACKGROUND OF THE INVENTION

Differentiation and proliferation of stem cells and progenitor cells are normal ongoing processes that act in concert to support tissue growth during organogenesis, cell repair and cell replacement. The system is tightly regulated to ensure that only appropriate signals are generated based on the needs of the organism. Cell proliferation and differentiation normally occur only as necessary for the replacement of damaged or dying cells or for growth. However, disruption of these processes can be triggered by many factors including the under- or overabundance of various signaling chemicals, the presence of altered microenvironments, genetic mutations or a combination thereof. Disruption of normal cellular proliferation and/or differentiation can lead to various disorders including proliferative diseases such as cancer.

Conventional therapeutic treatments for cancer include chemotherapy, radiotherapy and immunotherapy. Often these treatments are ineffective and surgical resection may not provide a viable clinical alternative. Limitations in the current standard of care are particularly evident in those cases where patients undergo first line treatments and subsequently relapse. In such cases refractory tumors, often aggressive and incurable, frequently arise. The overall survival rate for many solid tumors have remained largely unchanged over the years due, at least in part, to the failure of existing therapies to prevent relapse, tumor recurrence and metastasis. There remains therefore a great need to develop more targeted and potent therapies for proliferative disorders. The current invention addresses this need.

### SUMMARY OF THE INVENTION

In a broad aspect the present invention provides compounds comprising isolated antibody drug conjugates, which specifically bind to human DLL3 determinant and may be used to treat neurological malignancies (e.g., tumors of the brain and central nervous system). In certain embodiments the DLL3 determinant is a DLL3 protein expressed on such tumor cells while in other embodiments the DLL3 determinant is expressed on neural tumor initiating cells. In certain embodiments the disclosed compounds and compositions may be used to treat diffuse and/or low grade glioma. In selected aspects of the invention the DLL3 ADC will comprise rovalpituzumab tesirine (Rova-T, CAS Registry Number 1613313-09-9). In certain embodiments the invention comprises a pharmaceutical composition comprising an ADC as described above.

Other aspects of the invention are directed to methods of treating diffuse and/or low grade glioma. In another embodiment the invention comprises a method of reducing tumor initiating cells in a glial tumor cell population, wherein the method comprises contacting (e.g. in vitro or in vivo) a glial tumor initiating cell population with an ADC as described herein whereby the frequency of the tumor initiating cells is reduced. In one aspect, the invention comprises a method of delivering a cytotoxin to a neural cancer cell comprising contacting the cell with any of the above described ADCs.

In selected embodiments the present invention will comprise treating patients having DLL3 positive tumors that also exhibit IDH mutations. More particularly, the disclosed compounds and compositions may be used to treat DLL3 positive IDH mutated (R132H) low grade glioma. In certain aspects the mutated gene will comprise IDH1. In other embodiments the mutated gene will comprise IDH2. In yet other aspects the tumor may exhibit mutations in both IDH1 and IDH2.

The foregoing is a summary and thus contains, by necessity, simplifications, generalizations, and omissions of detail; consequently, those skilled in the art will appreciate that the summary is illustrative only and is not intended to be in any way limiting. Other aspects, features, and advantages of the methods, compositions and/or devices and/or other subject matter described herein will become apparent in the teachings set forth herein.

### BRIEF DESCRIPTION OF THE FIGURES

FIG. 1 shows RNA expression levels of DLL3 in as measured using whole transcriptome sequencing of normal brain tissue and brain tumors in TOGA;

FIGS. 2A and 2B depict, respectively, the relative protein expression levels of DLL3 as measured by immunohistochemistry in normal brain and brain tumors (low grade, astrocytoma, glioblastoma) and that DLL3 expression correlates with IDH mutated low grade glioma; and

FIG. 3 shows efficacy with an anti-DLL3 antibody drug conjugate in a glioblastoma PDX cell line.

### DETAILED DESCRIPTION OF THE INVENTION

Disclosed herein are non-limiting, illustrative embodiments of the invention that exemplify the principles thereof. Any section headings used herein are for organizational purposes only and are not to be construed as limiting the subject matter described. For the purposes of the instant disclosure all identifying sequence accession numbers may be found in the NCBI Reference Sequence (RefSeq) database and/or the NCBI GenBank® archival sequence database unless otherwise noted.

DLL3 expression has been found to correlate with certain neural malignancies including diffuse and/or low grade glioma. It has also unexpectedly been found that DLL3 expression is associated with glial tumorigenic cells and, as such, may be effectively exploited to inhibit or eliminate such cells. Tumorigenic cells, which will be described in more detail below, are known to exhibit resistance to many conventional treatments. In contrast to the teachings of the prior art, the disclosed compounds and methods effectively overcome this inherent resistance.

The invention provides rovalpituzumab tesirine (Rova-T) for use in the treatment of DLL3-associated neural malignancies. In this regard, Rova-T may be particularly useful for targeting tumorigenic cells thereby facilitating the treatment of cancer. Whether by inhibition or elimination of the tumorigenic cells, modification of their potential (for example, by induced differentiation or niche disruption) or otherwise interfering with the ability of tumorigenic cells to influence the tumor environment or other cells, the present invention allows for more effective treatment of cancer by inhibiting tumorigenesis, tumor maintenance and/or metastasis and recurrence. It will further be appreciated that the same characteristics of the disclosed antibodies make them particularly effective at treating recurrent tumors which have proved resistant or refractory to standard treatment regimens.

Methods that can be used to assess a reduction in the frequency of tumorigenic cells include, but are not limited to, cytometric or immunohistochemical analysis, preferably by in vitro or in vivo limiting dilution analysis (Dylla et al. 2008, PMID: PMC2413402 and Hoey et al. 2009, PMID: 19664991).

The ability of Rova-T to reduce the frequency of tumorigenic cells can therefore be determined using the techniques and markers known in the art. In some instances, Rova T may reduce the frequency of tumorigenic cells by 10%, 15%, 20%, 25%, 30% or even by 35%. In other embodiments, the reduction in frequency of tumorigenic cells may be in the order of 40%, 45%, 50%, 55%, 60% or 65%. In certain embodiments, the disclosed compounds may reduce the frequency of tumorigenic cells by 70%, 75%, 80%, 85%, 90% or even 95%. It will be appreciated that any reduction of the frequency of tumorigenic cells is likely to result in a corresponding reduction in the tumorigenicity, persistence, recurrence and aggressiveness of the neoplasia.

Rova-T is a first in class antibody drug conjugate directed to the tumor associated antigen delta-like ligand 3 (DLL3) that is currently in Phase III clinical studies. The drug comprises an anti-DLL3 antibody conjugated to a dimeric pyrrolobenzodiazepine (PBD) cytotoxin through a cleavable linker. To date Rova-T has clinically demonstrated single agent activity in recurrent/refractory small cell lung cancer (SCLC). See, for example, U.S. Pat. No. 9,968,687, Rudin et al: Lancet Oncol 18:42-51, 2017 and Saunders et al. Sci. Transl. Med. 2015; 7(302): 1-13, each of which is incorporated herein in its entirety by reference.

Rovalpituzumab tesirine may be represented by the following structure:

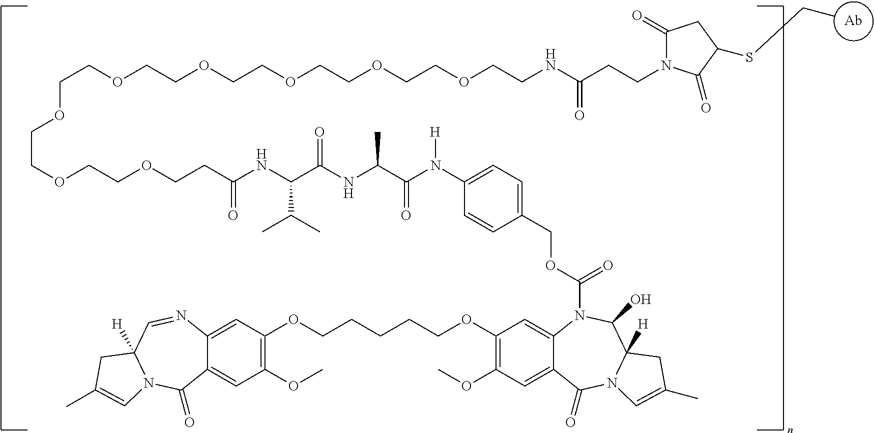

wherein Ab comprises a humanized anti-DLL3 antibody having a heavy chain of SEQ ID NO: 1 and a light chain of SEQ ID NO: 2 and wherein n is an integer from 1 to 8. Rova-T compositions comprise a consistent distribution of heterogeneous drug-load variants with a target average drug-to-antibody molar ratio (DAR) of approximately 2.

The full length heavy (SEQ ID NO: 1) and light (SEQ ID NO: 2) chain amino acid sequences for the humanized Rova-T antibody are set forth immediately below.

(SEQ ID NO: 1)
QVQLVQSGAEVKKPGASVKVSCKASGYTFTNYGMNWVRQAPGQGLEWMGW
INTYTGEPTYADDFKGRVTMTTDTSTSTAYMELRSLRSDDTAVYYCARIG
DSSPSDYWGQGTLVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKKVEPKSCDKTHTCPPCPAPELLGGPSVFLFPPKPKD
TLMISRTPEVTCVVVDVSHEDPEVKFNWYVDGVEVHNAKTKPREEQYNST
YRVVSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLD
SDGSFFLYSKLTVDKSRWQQGNVFSCSVMHEALHNHYTQKSLSLSPG.
(SEQ ID NO: 2)
EIVMTQSPATLSVSPGERATLSCKASQSVSNDVVWYQQKPGQAPRLLIYY
ASNRYTGIPARFSGSGSGTEFTLTISSLQSEDFAVYYCQQDYTSPWTFGQ
GTKLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC.

Those of skill in the art will appreciate that the aforementioned ADC structure is defined by the formula Ab-[L-D]n and more than one drug linker [L-D] molecule as depicted therein may be covalently conjugated to the DLL3 antibody (e.g., n may be an integer from 1 to 8). More particularly, it will be appreciated that more than one payload may be conjugated to each antibody and that the schematic representations above must be construed as such. By way of example compositions of Rova-T as set forth above may comprise a DLL3 antibody conjugated to 1, 2, 3, 4, 5, 6, 7 or 8 or more payloads and that compositions of such ADCs will generally comprise a mixture of drug loaded species.

In this regard the average DAR value represents the weighted average of drug loading for the composition as a whole (i.e., all the ADC species taken together). Due to inherent uncertainty in the quantification methodology employed and the difficulty in completely removing the non-predominant ADC species in a commercial setting, acceptable DAR values or specifications are often presented as an average, a range or distribution (i.e., an average DAR of 2+/−0.5). Preferably compositions comprising a measured average DAR within the range (i.e., 1.5 to 2.5) would be used in a pharmaceutical setting. It will be appreciated that the range or deviation may be less than 0.5 in some embodiments. Thus, in other embodiments the compositions will comprise an average DAR of 1, 2, 3, 4, 5, 6, 7 or 8 each +/−0.3, an average DAR of 2, 4, 6 or 8+/−0.3, even more preferably an average DAR of 2 or 4+/−0.3 or even an average DAR of 2+/−0.3. In other embodiments IgG1 conjugate compositions will preferably comprise a composition with an average DAR of 1, 2, 3, 4, 5, 6, 7 or 8 each +/−0.4

The ADCs of the invention can be formulated in various ways using art recognized techniques. In some embodiments, the therapeutic compositions of the invention can be administered neat or with a minimum of additional components while others may optionally be formulated to contain suitable pharmaceutically acceptable carriers. As used herein, “pharmaceutically acceptable carriers” comprise excipients, vehicles, adjuvants and diluents that are well known in the art and can be available from commercial sources for use in pharmaceutical preparation (see, e.g., Gennaro (2003) Remington: The Science and Practice of Pharmacy with Facts and Comparisons: Drugfacts Plus, 20th ed., Mack Publishing; Ansel et al. (2004) Pharmaceutical Dosage Forms and Drug Delivery Systems, 7^{th} ed., Lippencott Williams and Wilkins; Kibbe et al. (2000) Handbook of Pharmaceutical Excipients, 3^{rd} ed., Pharmaceutical Press.)

The invention provides for the use of Rova-T, for the treatment of neural proliferative disorders. In certain embodiments the diseases to be treated comprise neural neoplastic conditions and in certain other aspects comprise solid tumors. In other embodiments the diseases to be treated comprise neural or CNS malignancies including gliomas. Preferably the “subject” or “patient” to be treated will be human although, as used herein, the terms are expressly held to comprise any mammalian species. In other embodiments the subject may comprise a child or pediatric subject.

As used herein the term glioma will mean any tumors arising from the glial cells of the central nervous system. Three types of glial cells can produce tumors and gliomas are classified according to the type of glial cell involved in the tumor. In this regard types of glioma include astrocytomas (e.g., astrocytoma, anaplastic astrocytoma and glioblastoma), ependymomas, (e.g., anaplastic ependymoma, myxopapillary ependymoma and subependymoma) and oligodendrogliomas (oligodendroglioma, anaplastic oligodendroglioma and anaplastic oligoastrocytoma).

Gliomas are further categorized according to their grade, which is determined by pathologic evaluation of the tumor. Low grade gliomas [WHO grade II] are well-differentiated (not anaplastic); these tend to exhibit benign tendencies and portend a better prognosis for the patient. However, they have a uniform rate of recurrence and increase in grade over time so should be classified as malignant. High-grade [WHO grade III-IV] gliomas are undifferentiated or anaplastic; these are malignant and carry a worse prognosis. Of numerous grading systems in use, the most common is the World Health Organization (WHO) grading system for astrocytoma, under which tumors are graded from I (least advanced disease—best prognosis) to IV (most advanced disease—worst prognosis).

In particular the present invention provides for the treatment of diffuse and low grade gliomas (LGG). Diffuse gliomas represent about 80% of malignant brain tumors (PMID: 16932614). Previous classifications of these tumors were based upon histology, first according to microscopic similarity to a putative cell of origin (e.g., ependymomas, astrocytomas, oligodendromas, brainstem gliomas, mixed gliomas, or optic nerve gliomas), followed by staging as defined by the level of cellular differentiation and tumor aggressiveness (PMID: 17618441). In these histological classification schemes, glioblastomas are malignant astrocytomas with aggressive, invasive stage IV behavior resulting in median survival times under 14 months. Most patients with lower grade gliomas will progress to glioblastoma within ten years; hence glioblastomas could be further subclassified on clinicopathological grounds as primary (e.g. de novo) or secondary (e.g. arising through progression from lower grade gliomas) (PMIDs: 11550301, 25957782). Recent (e.g. 2016) revisions to the WHO classification system for tumors of the central nervous system (PMID: 27157931) have incorporated genotypic and molecular features, such as mutation status of the isocitrate dehydrogenase (IDH1/2) genes (PMID: 19228619). In particular, four molecular and cytogenetic markers have better captured related tumor subtypes for diffuse low grade gliomas (LGG) than histology alone: IDH mutation status, 1p/19q co-deletions, chromatin remodeler ATRX loss-of-function, and TP53 mutation (PMID: 29034211). Additionally, the 2016 revisions to the WHO classification scheme include a new entity, “diffuse midline glioma, H3 K27mutant,” to encompasses brainstem gliomas and diffuse intrinsic pontine gliomas (DIPG). These tumor types, more common in pediatric or young (<20) adult patients than in older adults, are not generally amenable to surgical treatment, and therefore like adult high-grade gliomas have dismal prognoses (median survival times <1 year). Although the precise molecular mechanisms relating the IDH, H3 K27M and ATRX mutations to LGG tumorigenesis and progression remain under intensive investigation, it is clear that each is linked to hypermethylation and dysregulated epigenetic modifications in the tumors.

Large-scale genomics analyses of bulk glioblastoma tumors differ in the absolute number of molecular and genetic subtypes they distinguish, but agree that a large subset (20-31%) possess a “pro-neural “gene signature, while in contrast, a second, large subset (33-49%) shows expression of genes associated with a mesenchymal phenotype (reviewed in PMID: 25957782). The proneural and mesenchymal expression signatures identified in glioblastoma also have intrinsic prognosis value in LGG, particularly the proneural signature in oligodendrogliomas (PMID: 20838435). Proneural expression signatures comprise many genes known to play a role in neurogenesis, including genes of the Notch pathway, specifically including Notch ligands such as DLL3. ASCL1 expression is higher in proneural tumors, correlates with higher tumor grade, and is essential for maintenance and propagations of GBM cancer stem cells (PMID: 23707066, 24726434). ASCL1 directly regulates the transcription of DLL3 (PMID: 19389376).

More detailed genomic and integrative analysis of grade II and grade III adult gliomas (PMIDs: 25848751, 26061751) have revealed three distinct subsets of low grade and intermediate-grade gliomas: (1) IDH mutant, 1p/19q deleted, TERT promoter mutant tumors with fairly positive prognosis, (2) IDH mutant, TP53 mutant tumors without 1p/19q co-deletions, and (3) IDH wild-type tumors, without 1p/19q co-deletion or TERT-promoter or TP53 mutants, having the poorest prognosis. Within the IDH wild-type tumors, grade-III tumors were linked to a median survival only slightly longer than that of primary glioblastoma (˜2 years), whereas the grade II tumors within the same subset showed a better prognosis (˜9 years). Mutations in the NOTCH1 gene were observed very infrequently (<10-15%) in adult gliomas, and were slightly more common in the IDH-mutant, 1p/19q co-deleted subset of tumors.

Pediatric gliomas, unlike their adult counterparts, infrequently show IDH-mutations. Instead, these tumors tend to have histone H3 mutations leading to aberrant transcription, including dysregulation of ASCL1 and NOTCH pathway genes (PMID: 28434841). DIPG also have characteristic copy number changes, involving several MYC family genes. Amplification of DLL3 has been reported in some DIPG samples (PMID: 20570930).

The dismal prognoses for adult and pediatric gliomas demonstrate the need for the development of novel therapeutics for the treatment of these tumors. The genomic analyses of these tumors described above suggest that subsets of patients presenting with these tumors might be responsive to a DLL3-targeted therapeutic, including Rova-T, provided they have expression of DLL3. Tumors with loss of 19q might be expected to show reduced expression of DLL3, given its location at chr19q13.2, but aberrant histone modification or constitutive Notch signaling might be expected to increase DLL3 expression, so direct measurement of DLL3 protein is likely the best way to determine if a patient would be a candidate for a DLL3-targeted therapeutic.

### EXAMPLES

The invention, thus generally described above, will be understood more readily by reference to the following examples, which are provided by way of illustration and are not intended to be limiting of the instant invention. The examples are not intended to represent that the experiments below are all or the only experiments performed. Unless indicated otherwise, parts are parts by weight, molecular weight is weight average molecular weight, temperature is in degrees Centigrade, and pressure is at or near atmospheric.

### Example 1

### DLL3 Expression in Low Grade Glioma and Glioblastoma Tumors from the Cancer Genome Atlas

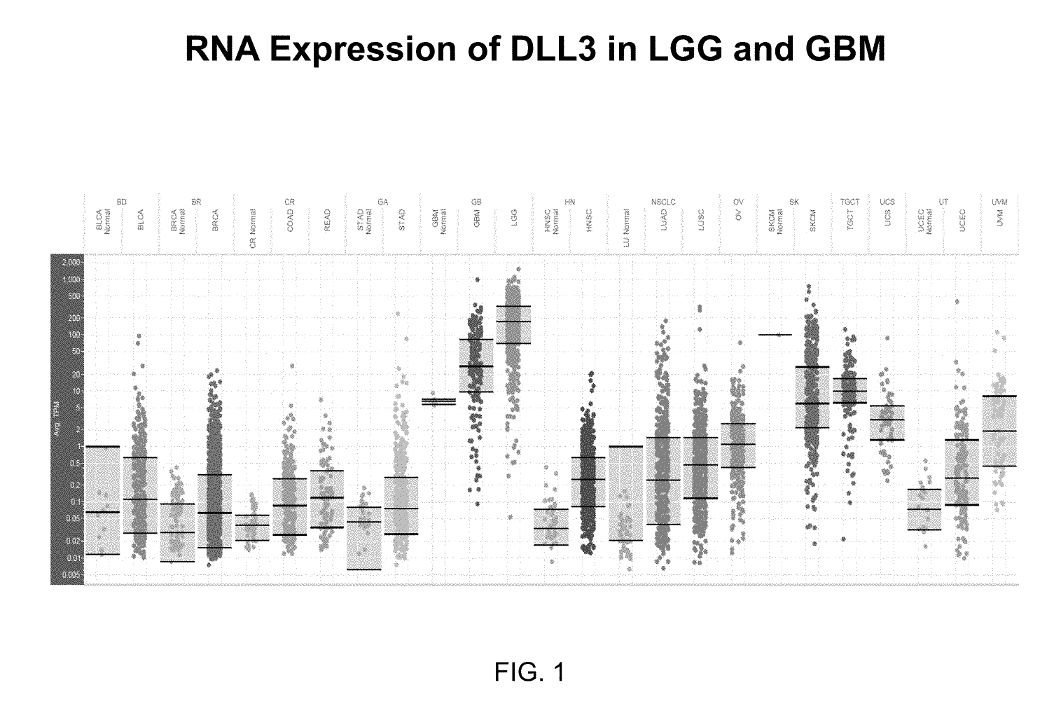

Overexpression of DLL3 mRNA in Low Grade Glioma (LGG) and Glioblastoma (GBM) tumors was identified using a large, publicly available dataset of tumor and normal samples known as The Cancer Genome Atlas (TCGA). DLL3 expression data from the IlluminaHiSeq_RNASeqV2 platform was downloaded from the TCGA Data Portal (https://tcga-data.nci.nih.gov/tcga/tcgaDownload.jsp), and parsed to aggregate the reads from the individual exons of each gene to generate a single value transcript per million mapped reads (TPM). FIG. 1 shows DLL3 expression is substantially elevated in most LGG and the majority of GBM tumors relative to normal brain and other tissues found in the TCGA database. In contrast, very low rpkm levels in normal breast, kidney, colon, lung and prostate tissue demonstrate the lack of DLL3 expression. These data confirm the previous observations that elevated DLL3 mRNA can be found in many LGG and GBM tumors but not in normal tissues, implying there is a good therapeutic index above normal tissues and therefore anti-DLL3 antibodies and ADCs may be useful therapeutics for these tumors.

### Example 2

### Detection of DLL3 Expression in Low Grade Glioma and Glioblastoma Tumors Using Immunohistochemistry

Using DLL3 specific antibodies immunohistochemistry (IHC) was performed on normal brain, low and high grade glioma tumor tissue sections to assess the expression and location of DLL3 normal brain and brain tumor cells.

In order to identify IHC-compatible anti-DLL3 antibodies, immunohistochemistry was performed on HEK-293T parental cell pellets or DLL3-expressing HEK-293T cell pellets using numerous anti-DLL3 antibodies generated as described herein. IHC was performed on HEK-293T cells pellets that were formalin fixed and paraffin embedded (FFPE) as is standard in the art. Planar sections of cell pellet blocks were cut and mounted on glass microscope slides. After xylene deparaffinization, 5 μm sections were pre-treated with Antigen Retrieval Solution (Dako) for 20 minutes at 99° C., cooled to 75° C. and then treated with 0.3% hydrogen peroxide in PBS followed by Avidin/Biotin Blocking Solution (Vector Laboratories) for 15 min at room temperature. FFPE slides were then blocked with 10% horse serum in 3% BSA in PBS buffer and incubated with a primary monoclonal anti-DLL3 antibody of the invention, diluted to 10 μg/ml in 3% BSA/PBS, for 60 minutes at room temperature. FFPE slides were incubated with biotin-conjugated horse anti-mouse antibody (Vector Laboratories), diluted to 2.5 μg/ml in 3% BSA/PBS, for 30 minutes at room temperature followed by incubation in streptavidin-HRP (ABC Elite Kit; Vector Laboratories) for 30 min at room temperature. After washing slides, chromogenic detection was developed with 3,3′-diaminobenzidine (Thermo Scientific) for 5 minutes at room temperature and tissues were counterstained with Meyer's hematoxylin (IHC World), washed with alcohol and immersed in xylene to coverslip and evaluate under microscope. Antibodies showing specific staining on DLL3-expressing HEK-293T cells but no staining on HEK-293T parent cell were selected and further evaluated for sensitivity and specificity using endogenous cell lines and DLL3 CRISPR knockdown cell lines.

The DLL3 IHC experiments on tumor tissue were carried out using Ventana's BenchMark ULTRA staining platform. Antigen recovery was conducted using Protease II (VMSI, Catalog No. 760-2019) for 16 minutes at 37° C. Slides were incubated with mouse anti-human DLL3 antibody (clone sc16.65) at 0.78 μg/ml of the final concentration for 32 minutes at 36° C. The anti-DLL3 antibody was detected using the OptiView™ detection kit (VMSI, Catalog No. 760-700). Tissues stained with anti-DLL3 show cytoplasmic and membrane staining. Tissues were scored on a percentage of tumor cells positive as well as a 0-3+ scale where 0, 1+, 2+, and 3+ indicate no DLL3 staining, weak, moderate and strong staining intensities respectively.

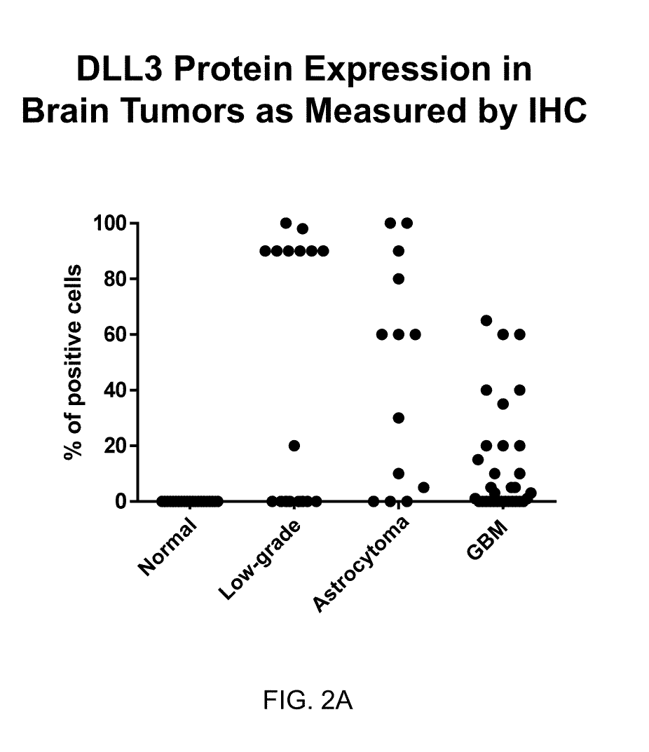

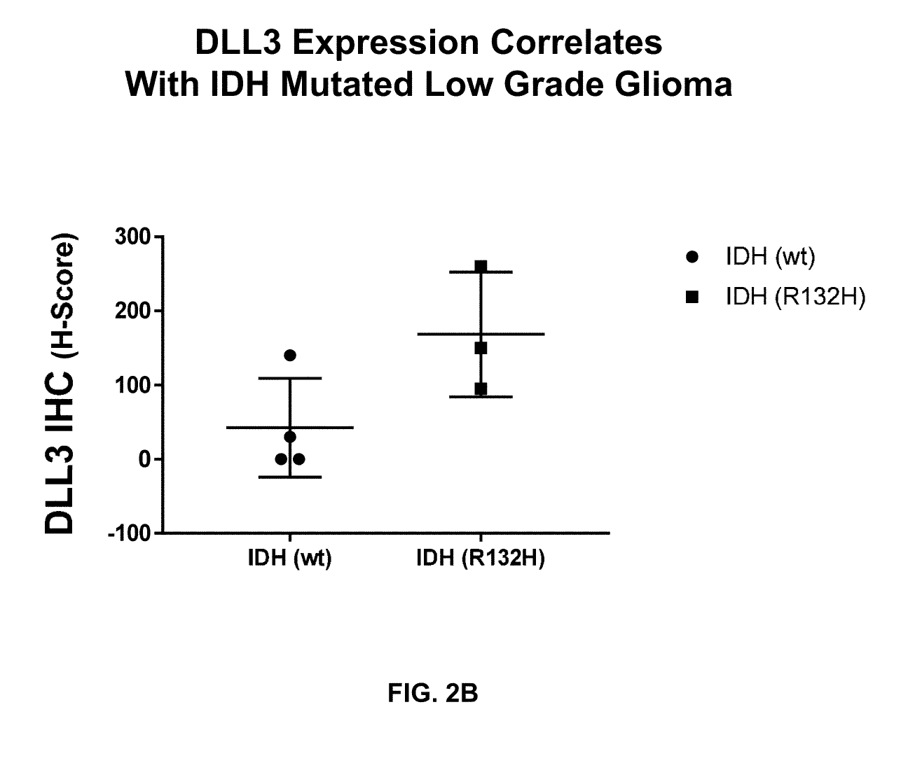

More specifically, the aforementioned procedure was used to determine whether DLL3 was expressed in various primary biopsies from normal brain, low grade glioma, astrocytoma and glioblastoma. The percentage of cells that expressed DLL3 was determined. FIG. 2A demonstrates that DLL3 protein expression was not seen in normal brain, was seen in 9/17 (53%) LGG, 3/6 (50%) astrocytomas, and 20/33 (60%) GBM. While approximately 50% of the primary brain tumors tested expressed DLL3 protein, the number of tumor cells that were positive decreased with higher grade tumors. These data demonstrate that anti-DLL3 antibodies have diagnostic and therapeutic utility in malignant brain tumors.

To determine if DLL3 expression is correlated with IDH mutant low grade glioma, DLL3 expression was compared to LGG with mutant IDH (R132H) detected by IHC. As shown in FIG. 2B, DLL3 expression is higher in IDH mutated (R132H) LGG. These data demonstrate that patients with IDH mutated LGG would benefit from anti-DLL3 therapeutic strategies.

### Example 3

### Anti-DLL3 Antibodies Suppress In Vivo Glioblastoma Growth

To illustrate the scope of the instant invention an anti-DLL3 ADC was tested to demonstrate its ability to kill and suppress glioblastoma tumor (GBM) growth in immunodeficient mice.

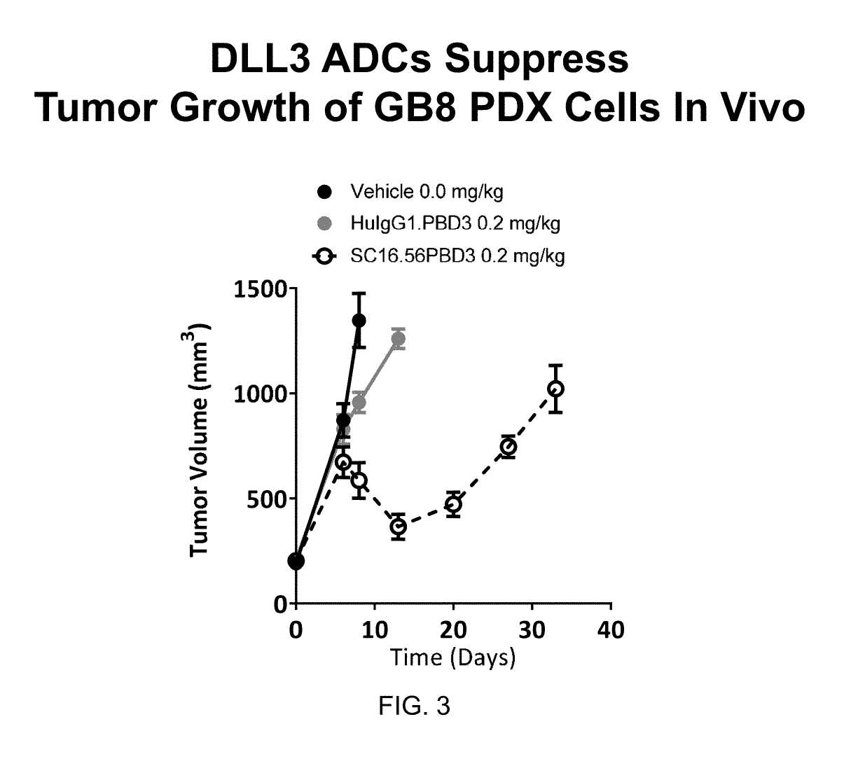

A GBM PDX tumor line was grown subcutaneously in the flanks of female NOD/SCID mice using art-recognized techniques. Tumor volumes and mouse weights were monitored once or twice per week. When tumor volumes reached 150-250 mm³, mice were randomly assigned to treatment groups and injected intraperitoneally with SC16.56 PDB3 or an isotype control human IgG1.PDB3. SC16.56 PDB3 comprises the same humanized antibody as found in Rova-t but employs a different PBD cytotoxin. Following treatment, tumor volumes and mouse weights were monitored until tumors exceeded 1000 mm³ or mice became sick. Mice treated with SC16.56 PBD3 did not exhibit any adverse health effects beyond those typically seen in immunodeficient, tumor-bearing NOD/SCID mice. Mice bearing GB8 tumors were given a single dose of SC16.56 PBD3, at 0.2 mg/kg, which resulted in tumor suppression 20 days post-treatment (FIG. 3).

The ability of SC16.56 PBD3 to suppress growth of DLL3-expressing glioblastoma tumor cells in vivo further validates the use of anti-DLL3 ADCs in the therapeutic treatment of human neural malignancies.

The complete disclosure of all patents, patent applications, and publications, and electronically available material (including; for example, nucleotide sequence submissions in, e.g., GenBank and RefSeq, and amino acid sequence submissions in, e.g., SwissProt, PIR, PRF, PBD, and translations from annotated coding regions in GenBank and RefSeq cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

1. A method of treating low grade glioma or diffuse glioma in a subject in need thereof comprising the step of administering rovalpituzumab tesirine.

2. The method of claim 1 wherein the glioma comprises low grade glioma.

3. The method of claim 1 wherein the glioma comprises diffuse glioma.

4. The method of claim 1 wherein the glioma comprises IDH mutated glioma.

5. The method of claim 4 wherein the IDH mutated glioma comprises IDH1 mutated glioma.

6. The method of claim 4 wherein the IDH mutated glioma comprises IDH2 mutated glioma.

7. The method of claim 1 wherein the glioma comprises a 1p/19q deletion glioma.

8. The method of claim 1 wherein the glioma comprises a TP53 mutant glioma.

9. The method of claim 1 wherein the glioma comprises a TERT-promoter mutant glioma.

10. The method of claim 1 wherein the glioma comprises an IDH wild-type glioma.

11. The method of claim 1 wherein the glioma is a pediatric glioma.

12. The method of claim 1 further comprising the step of contacting the glioma with an DLL3 detection agent and detecting the DLL3 detection agent associated with the glioma.

13. A method of treating low grade glioma or diffuse glioma in a subject in need thereof comprising the step of administering a DLL3 antibody drug conjugate (DLL3 ADC) wherein the DLL3 ADC comprises the structure: 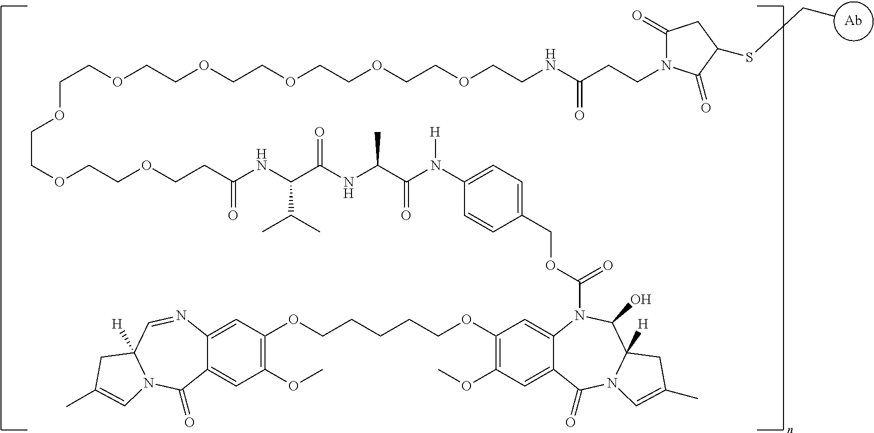
wherein Ab comprises an anti-DLL3 antibody having a heavy chain amino acid sequence of SEQ ID NO: 1 and a light chain amino acid sequence of SEQ ID NO: 2 and wherein n is an integer from about 1 to about 8.

14. The method of claim 13 wherein the glioma comprises low grade glioma.

15. The method of claim 13 wherein the glioma comprises diffuse glioma.

16. The method of claim 13 wherein n is 2.

17. A method of treating low grade or diffuse glioma in a patient in need thereof comprising the step of administering a pharmaceutical composition wherein the pharmaceutical composition comprises:
a. rovalpituzumab tesirine; and
b. a pharmaceutically acceptable carrier.

18. The method of claim 17 wherein the glioma comprises low grade glioma.

19. The method of claim 17 wherein the glioma comprises diffuse glioma.

20. The method of claim 17 wherein the pharmaceutical composition comprises an average drug to antibody ratio (DAR) of 2±0.3.


SEQUENCE LISTING (2 sequences)

SEQ ID NO: 1 - protein, 447 aa, Artificial Sequence
  Humanized full-length Rova-T antibody heavy chain
QVQLVQSGAEVKKPGASVKVSCKASGYTFTNYGMNWVRQAPGQGLEWMGWINTYTGEPTYADDFKGRVTMTTDTSTSTAYMELRSLRSDDTAVYYCARIGDSSPSDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 2 - protein, 214 aa, Artificial Sequence
  Humanized full-length Rova-T antibody light chain
EIVMTQSPATLSVSPGERATLSCKASQSVSNDVVWYQQKPGQAPRLLIYYASNRYTGIPARFSGSGSGTEFTLTISSLQSEDFAVYYCQQDYTSPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC